(19) United States
(12) Patent Application Publication
(10) Pub. No.: US 2024/0325318 A1
(43) Pub. Date: Oct. 3, 2024

(54) SOLUBILIZATE WITH AT LEAST ONE CANNABINOID

(71) Applicant: Aquanova AG, Darmstadt (DE)

(72) Inventor: Dariush BEHNAM, Rossdorf (DE)

(73) Assignee: Aquanova AG, Darmstadt (DE)

(21) Appl. No.: 18/738,127

(22) Filed: Jun. 10, 2024

Related U.S. Application Data
(63) Continuation of application No. 17/258,363, filed on Jan. 6, 2021.

(30) Foreign Application Priority Data
Jul. 11, 2018 (WO) PCT/EP2018/068731

(51) Int. Cl. A61K 31/12; A61K 9/107; A61K 9/48; A61K 31/05; A61K 31/122; A61K 31/19; A61K 31/352; A61K 36/324; A61K 38/48; A61K 47/10; A61K 47/26

(52) U.S. Cl. CPC A61K 31/12; A61K 9/107; A61K 9/1075; A61K 9/48; A61K 9/4825; A61K 31/05; A61K 31/122; A61K 31/19; A61K 31/352; A61K 36/324; A61K 38/4886; A61K 47/10; A61K 47/26

(57) ABSTRACT

In order to make available the health-promoting and healing properties of cannabinoids to the human organism, a solubilizate comprises, and in particular consists of, at least one cannabinoid in a content of less than or equal to 10 wt. % and at least one emulsifier having an HLB value in a range of less than 18, preferably between 13 and 18, in particular polysorbate 80 or polysorbate 20 or at least one sucrose ester of edible fatty acids.

### CROSS-REFERENCE TO RELATED APPLICATION

This application is a continuation of application U.S. 17/258,363, filed on Jan. 6, 2021, which is a national stage application of International Patent Application PCT/EP2019/068537, filed on Jul. 10, 2019, and claims the benefit of European Patent Application PCT/EP2018/068731, filed on Jul. 11, 2018.

### BACKGROUND

Curcumin is discussed as an active substance based on various potential pharmacological properties. For example, there are indications for the antioxidant and also for the anti-inflammatory effect of curcumin as well as for the effectiveness against viruses and bacteria and against cancer. Indications could therefore be, for example, Parkinson's, Alzheimer's, diabetes, colorectal tumors, pancreatic cancer, and liver dysfunction.

Cannabinoids are transformation products and synthetic analogues of some terpene phenols that are mainly found in hemp plants (Cannabis).

The term “hemp plant” or “cannabis plant” encompasses the wild type Cannabis sativa and also variants thereof, including Cannabis chemovar, which naturally contain different amounts of the various cannabinoids, Cannabis sativa subspecies indica, and also plants that are the result of genetic crosses, self-crosses, or hybrids thereof.

The cannabis plant contains at least 113 cannabinoids from the group of terpene phenols. These cannabinoids include, inter alia, 49-tetrahydrocannabinol (THC), 48-tetrahydrocannabinol, cannabidiol (CBD), cannabichromene (CBC), cannabigerol (CBG), cannabinol (CBN), 49-tetrahydrocannabivarine (THCV), cannabicyclol (CBL), Cannabielsoin (CBE), cannabitriol (CBT), and cannabinodiol (CBND).

Tetrahydrocannabinol (THC) as a psychoactive substance is thought to mainly cause the psychedelic effect of cannabis products. The plant naturally contains THC in the form of two THC acids which are converted into THC by heating the plant material.

In addition to the psychedelic effect, THC is ascribed considerable medical properties such as pain-relieving and relaxing, appetizing, antioxidant, and neuroprotective effects as well as alleviating glaucoma.

Cannabidiol (CBD) is a non-psychoactive cannabinoid which also has antispasmodic, anti-inflammatory, anti-anxiety, immunosuppressive, neuroprotective and antioxidant effects and an anti-nausea effect, inter alia. In addition, CBD is thought to lower high blood sugar levels and stimulate bone formation. A synergistic effect can arise together with THC.

Like CBD, cannabichromene (CBC) does not have a psychedelic effect. It is in particular thought to have an antifungal, antidepressant effect. Cannabinol (CBN) is an oxidation product of THC and has a psychedelic effect. In addition to analgesic, relaxing, and calming effects, it is thought to have an appetizing and antibacterial, anti-inflammatory, and anti-asthmatic effect. Cannabigerol (CBG) is not psychoactive and is said to in particular have a greater analgesic effect than THC. Tetrahydrocannabivarin (THCV) has a lower psychoactive effect compared to THC and has an appetite-suppressing and metabolism-stimulating effect, inter alia.

Cannabis furthermore contains a variety of non-cannabinoids with diverse pharmacological properties. There are some indications that cannabinoids such as cannabinol (CBN), cannabidiol (CBD) and others modify the effects of 49-THC.

Synthetic cannabinoids may be produced semi-synthetically, i.e. from natural cannabinoids, or else fully synthetically from simple raw materials. There are also other plants that can produce active substances which are effective in the human body through the same mechanisms as the cannabinoids of the hemp plant.

In the context of the present application, the term “cannabinoid” encompasses not only natural and synthetic cannabinoids, but also phytocannabinoids. These include, for example, N-isobutylamides from Echinacea, and beta-caryophyllene which is contained in various aromatic plants, as well as yangonin from the kava plant (Piper methysticum), and various catechins from the tea plant (Camellia sinensis).

In order to be able to enter the bloodstream after oral intake, the active substance must pass through the small intestinal blood barrier, is then metabolized in the liver and enters the hepatic vein as a bioavailable fraction. The rest of the total active substance ingested and released in the body is either degraded microbially in the intestine or eliminated with the faeces or bile.

A toxicity due to the micellization of the active substance according to the invention in comparison to the native form could be ruled out on the basis of studies with MTT assays for cell viability. The verification of cell vitality by MTT assay is based on the reduction of the yellow water-soluble dye 3-(4,5-dimethylthiazol-2-yl)-2,5-diphenyltetrazolium bromide (MTT) into a blue-violet water-insoluble formazan.

### SUMMARY

An object of the disclosure is to provide nutritionally and/or pharmacologically acceptable cannabinoids. More particularly, it is an object of the invention to provide cannabinoids in a form that, when mixed with water, give a mixture free of turbidity for the human eye.

This is achieved by a solubilizate comprising, in particular consisting of at least one cannabinoid, in particular CBD and/or THC, in a content of less than or equal to 10 wt. %, preferably less than or equal to 5 wt. %, most preferably 0.3 wt. % to 3 wt. %; and at least one emulsifier having an HLB value in a range below 18, preferably between 13 and 18, in particular polysorbate 80 or polysorbate 20 or at least one sucrose ester of edible fatty acids, or a mixture of at least two emulsifiers selected from the group comprising polysorbate 20, polysorbate 80, and sucrose esters of edible fatty acids.

Such a solubilizate can be used in mixtures with curcumin solubilizates. These may also contain further active substances. The cannabinoid solubilizate per se moreover offers the advantage of providing cannabinoids in a form that can be administered orally, for example in beverages or as a filling of capsules. The solubilization of the cannabinoid, in particular the CBD, provides an efficient protection against oxidation for this active substance. In particular CBD is otherwise prone to oxidation, which means that the active substance content might decrease during the shelf life of CBD-containing products. This is prevented by the invention. With the improved oxidative protection of the cannabinoid active substance, the invention permits to provide products with a longer shelf life.

In an advantageous embodiment, the solubilizate is provided in a form that can be administered orally, in particular in a curcumin dose in the range from 0.5 mg/kg body weight to 1 mg/kg body weight, preferably in a dose of 0.81 mg/kg body weight, in particular once a day.

For the purposes of the present application, the term “active substance” refers to a substance that is provided in a pharmaceutically effective concentration and is preferably added for the purpose of having a pharmaceutical effect. Here, the name of the respective active substance is understood to encompass also substances that are converted in the body into the active substance and/or into its biologically active form.

One active substance in the context of the present application is a cannabinoid. “Active substances” in the sense of the present application include secondary phytochemicals which are produced as chemical compounds by plants, neither in energy metabolism nor in anabolic or catabolic metabolism. One group of secondary phytochemicals and thus active substances in the sense of the present application are flavonoids. The active substances in the sense of the present application also include natural polyphenols such as resveratrol or the polyphenols from licorice, and natural phenols, in particular chalcones such as xanthohumol. Also encompassed are plant extracts, i.e. substances that were extracted from plants or parts of plants using an extractant. These include extracts from hops. The active substance is referred to as an “extract” even if it is still dissolved in the extractant. Also the term “essence” can be used for an extract.

The “active substances” in the sense of the present application also include enzymes. One example of an enzyme as an “active substance” in the present application is serrapeptase. However, the application is not limited to this enzyme.

The extract from the resin of the frankincense tree, Boswellia serrata extract, contains several pentacyclic triterpenes which together are often referred to as total boswellic acids (“total BAs”). The term “boswellic acids” refers to a group of chemical compounds naturally occurring in the resin of the frankincense trees mentioned above. The two basic structures are a-boswellic acid and β-boswellic acid. Also, some derivatives of the boswellic acids are known, in particular compounds which carry a keto group at position 11 and/or which are acetylated at position 3. Boswellic acids that are currently considered to be significant in terms of pharmacological effects in particular include α-boswellic acid (αBA) and β-boswellic acid (BBA) and their derivatives 11-keto-β-boswellic acid (KBA); CAS 17019-92-0) and 3-O-acetyl-11-keto-β-boswellic acid (AKBA); CAS 67416-16-9), and 3-O-acetyl-α-boswellic acid (AαBA), and 3-O-acetyl-β-boswellic acid (AβBA). In particular the derivative AKBA is considered to have an anti-inflammatory effect.

In the context of the present application, the term “Boswellia”, in particular in the term “Boswellia solubilizate” is used in the sense that the term “Boswellia” refers to the active substances from the resin of the frankincense tree, i.e. to at least one boswellic acid and/or at least one derivative of a boswellic acid. The term “boswellic acid solubilizate” refers to a micellar formulation of at least one boswellic acid which may also contain at least one boswellic acid derivative.

Xanthohumol is a flavonoid naturally occurring in hops. It is a prenylated plant polyphenol which is assigned to the chalcones and has only been identified in hops so far. The bitter hop varieties have a significantly higher content of xanthohumol than aroma varieties. In tests, xanthohumol was found to be effective against the emergence and development of cancer cells. In laboratory experiments, it was moreover found that xanthohumol is capable of protecting the nerve cells of the brain and thus could possibly help to slow down the course of diseases like Alzheimer's or Parkinson's.

Serratia peptidase or serrapeptase is a proteolytic enzyme produced by the bacterium Serratia which lives in the intestine of the silkworm. Serrapeptase is said to have beneficial effects in relieving pain, inflammation, traumatic swelling, and excess mucus secretion by the organism. It is said to be effective like an anti-inflammatory and analgesic similar to acetylsalicylic acid, ibuprofen or other non-steroidal analgesics. It is also said to induce fibrinolytic anti-inflammatory and anti-oedematous activity in the tissue. Like all enzymes, serrapeptase is sensitive to the acids produced by the stomach. Therefore, the provision in a formulation that allows gastric passage is an object of the disclosure.

The solubilizate may contain one or more boswellic acids and/or one or more boswellic acid derivatives in a content of less than or equal to 10 wt. %, preferably less than or equal to 5 wt. %, most preferably 2 wt. % to 4 wt. %.

Due to the high proportion of Boswellia, the solubilizate may contain an extract obtained from the resin of the plant Boswellia serrata by extraction using ethyl acetate, as a source of the one or more boswellic acids and/or one or more boswellic acid derivatives, with boswellic acids being contained in a concentration of at least 85 wt. % in this extract.

The solubilizate may contain xanthohumol in an amount of less than or equal to 10 wt. %, preferably less than or equal to 5 wt. %, most preferably 1 wt. % to 3 wt. %.

Due to the high proportion of xanthohumol, the solubilizate may contain an ethanolic extract of hard resins from hops as a source of xanthohumol, with a xanthohumol concentration in this extract in a range between 65 wt. % and 95 wt. %, preferably in a concentration in a range from 80% to 92 wt. %. In particular the products “Xantho-Flav Pure” or “Xantho-Flav” that will be discussed in more detail below can be used as a xanthohumol source in the context of the disclosure. The solubilizate may contain serrapeptase in a range of up to 3 wt. %, preferably in a range between 0.1 wt. % and 2 wt. %, most preferably in a range between 0.18 wt. % and 0.35 wt. %.

Also a solubilizate consisting of or comprising curcumin and at least one cannabinoid as a further active substance may also be provided or employed advantageously for use as an antibiotic and/or as a pharmaceutical drug in the treatment and/or prevention of diseases involving inflammation, cancer, Alzheimer's, Parkinson's, obesity, high cholesterol, elevated blood sugar, diabetes, metabolic syndrome, and/or autoimmune diseases, multiple sclerosis (MS), for reducing visceral fat, for thermogenesis, for lowering cholesterol, in particular LDL cholesterol, and/or glucose in the blood and/or triglycerides in the blood, for improving macular pigment density, for reducing oxidative stress and/or for reducing the accumulation of fat in the hepatocytes, in particular as a pharmaceutical drug for treating and/or preventing fatty liver disease, Friedreich's ataxia, lysosomal diseases, in particular Tay-Sachs disease, arteriosclerosis, heart diseases, arthritis.

In particular, the solubilizates as described above may be uzed as an anti-inflammatory drug and/or as an antibiotic and/or as a pharmaceutical drug with an effect against cancer, Alzheimer's, Parkinson's, obesity, high cholesterol, elevated blood sugar, diabetes, metabolic syndrome, and/or autoimmune diseases, multiple sclerosis (MS), for lowering visceral fat, for thermogenesis, as a cholesterol-lowering pharmaceutical drug, in particular with respect to LDL cholesterol, and/or as a pharmaceutical drug with an effect for lowering glucose in the blood and/or triglycerides in the blood, for improving macular pigment density, for reducing oxidative stress and/or for reducing the accumulation of fat in the hepatocytes, in particular as a pharmaceutical drug with an effect against fatty liver, Friedreich's ataxia, lysosomal diseases, in particular Tay-Sachs disease, arteriosclerosis, heart diseases, arthritis.

Depending on the further components that are contained in the solubilizate, in order to provide stable micelles of the active substances, the emulsifier content, in particular the polysorbate content, may be at least 70 wt. %, preferably in the range between 75 wt. % and 95 wt. %, most preferably in the range between 79 wt. % and 88 wt. %.

Depending on the specific application case, the solubilizate may contain up to 20 wt. %, preferably up to 15 wt. % of ethanol, for example, and/or up to 25 wt. %, preferably between 12 wt. % and 20 wt. %, most preferably up to 10 wt. % of glycerol, and/or additionally up to 10 wt. %, preferably up to 7 wt. % of water. The addition of ethanol allows to reduce the content of emulsifier, in particular the content of polysorbate, which is an advantage in view of the ADI value for polysorbate (25 mg/kg body weight) as recommended by WHO. The content of emulsifier, in particular the content of polysorbate may also be reduced by adding glycerol.

The solubilizates have a narrow particle size distribution with small mean particle sizes, even under the physiological conditions of a gastric passage; the diameter distribution of the micelles in a dilution of the solubilizate with distilled water in a ratio of 1:500 at pH 1.1 and 37°° C. ranges from about d₁₀=6 nm to about d₉₀=20 nm. These values were determined from a volume distribution. Details of particle size analysis of the micelles of the solubilizates will be explained below.

An indication of the improved bioavailability compared to compositions of at least one cannabinoid or of curcumin and at least one cannabinoid as a further active substance that have not been micellated is obtained by a measurement of turbidity of the solubilizate, which is much easier accessible to measurement techniques. As a result of the formulation according to the disclosure, the turbidity of the solubilizate is preferably less than 25 FNU, more preferably less than 3 FNU, as measured by scattered light measurement using infrared light in compliance with the specifications of the ISO 7027 standard at a dilution of the solubilizate in a ratio of 1:50 in water under physiological conditions (pH 1.1 and 37° C.).

In order to facilitate oral application of a solubilizate in a more simple and convenient way for the consumer or patient, the disclosure also provides a capsule filled with a solubilizate as described above, wherein the capsule is in the form of a soft gelatin capsule or a hard gelatin capsule or a soft gelatin-free capsule or a hard gelatin-free capsule, for example a cellulose capsule.

Moreover, the solubilizate may be incorporated into other fluids, in particular liquids. The active substance-filled small micelles will be retained when doing so. Thus, the disclosure also provides a fluid containing the solubilizate as described above, wherein the fluid is selected from the group comprising foods, dietary supplements, beverages, cosmetics, and pharmaceutical products. The fluid may in particular comprise an aqueous dilution of the solubilizate.

The disclosure furthermore provides a method for treating and/or preventing diseases involving inflammation, cancer, Alzheimer's, Parkinson's, obesity, high cholesterol, elevated blood sugar, diabetes, metabolic syndrome, and/or autoimmune diseases, multiple sclerosis (MS), for reducing visceral fat, for thermogenesis, for lowering cholesterol, in particular LDL cholesterol, and/or glucose in the blood and/or triglycerides in the blood, for improving macular pigment density, for reducing oxidative stress and/or for reducing the accumulation of fat in the hepatocytes, in particular as a pharmaceutical drug for treating and/or preventing fatty liver disease, Friedreich's ataxia, lysosomal diseases, in particular Tay-Sachs disease, arteriosclerosis, heart diseases, arthritis, for improving macular pigment density, wherein a solubilizate, in particular in a capsule or as a fluid, is administered to the dietary supplement consumer or patient in particular orally, in particular once a day.

For producing a solubilizate comprising curcumin and at least one cannabinoid as a further active substance, it is possible to either mix together individually prepared solubilizates, or to directly prepare a solubilizate that contains curcumin and at least one cannabinoid as the further active substance.

The disclosure furthermore provides a method for producing a solubilizate as described above. If co-micellization of curcumin and at least one cannabinoid as a further active substance is desired, the following first variant of a preparation method includes the following steps
(a) providing polysorbate 80 and/or polysorbate 20 and/or at least one sucrose ester of edible fatty acids and/or a mixture of emulsifiers selected from the group comprising polysorbate 20, polysorbate 80, and sucrose esters of edible fatty acids;
(b) adding the at least one cannabinoid, in particular in the form of CBD oil and/or THC oil;
(c) adding curcumin powder;

wherein step (a) comprises heating to a temperature in the range from 40° C. to 62° C., preferably to a temperature in the range from 45° C. to 57° C., most preferably to a temperature in the range between 48° C. and 52° C.;

and wherein step (b) comprises heating to a temperature in the range from 82° C. to 97° C., preferably to a temperature in the range from 83° C. to 92° C., most preferably to a temperature in the range from 85° C. to 89° C.;
wherein this temperature is maintained in step (c).

This preparation method allows to produce a solubilizate which, in an aqueous dilution, is able to form micelles loaded with curcumin and with at least one cannabinoid as a further active substance. For this purpose, it is also possible to mix the at least two active substances with one another in a preparatory step under appropriately adapted temperature control, and then to add them in combined form, as a mixture.

In the context of the disclosure, the at least one cannabinoid is in particular used in the form of CBD oil, THC oil, or a mixture of the two. In principle, any formulation can be used as a source of cannabinoids. For example it is possible to use oils, in particular those known as “full-spectrum” oils, pastes, powders, crystalline forms, and/or isolates, with all of the formulations mentioned containing at least one cannabinoid. In principle, it is also possible to use extracts of at least one cannabinoid in order to obtain a solubilizate. The formulations mentioned may be those that essentially contain one cannabinoid, for example CBD in what is known as “CBD oil”. However, it is also possible to provide mixtures of at least two cannabinoids in a cannabinoid formulation that is used as a cannabinoid.

During step (b), also further active substances such as for example Boswellia serrata extract and/or xanthohumol may be incorporated.

Additionally or alternatively, further active substances such as for example serrapeptase may be incorporated in step (c). In addition or as an alternative to further active substances, MCT oil can be added in step (c).

In particular in this case, before step (b) a step
(b1) adding water at a temperature in the range from 40° C. to 62° C., preferably at a temperature in the range from 45° C. to 57° C., most preferably at a temperature in the range from 48° C. to 52° C.
   is carried out.

Additionally or alternatively, in step (b1) also the addition of ethanol can be carried out at a temperature in the range from 40° C. to 62° C., preferably at a temperature in the range from 45° C. to 57° C., most preferably at a temperature in the range from 48° C. to 52° C.

The disclosure also relates to solubilizates which exhibit in aqueous dilution both micelles loaded with curcumin alone or else with another active substance alone, at least immediately after their preparation. Therefore, the disclosure also provides a method for producing a solubilizate as described above by mixing a curcumin solubilizate and at least one cannabinoid solubilizate, in particular in a quantitative ratio of 1:1 of the individual solubilizates.

To this end, the disclosure provides for a method for producing a cannabinoid solubilizate with the following steps:
(a) providing polysorbate 80 and/or polysorbate 20 and/or at least one sucrose ester of edible fatty acids and/or a mixture of emulsifiers selected from the group comprising polysorbate 20, polysorbate 80, and sucrose esters of edible fatty acids;
(b) adding the at least one cannabinoid, for example in the form of CBD oil and/or THC oil;

wherein in step (a) a heating to a temperature at least in a range from 40° C. to 62° C., preferably to a temperature in a range from 45° C. to 57° C., most preferably to a temperature in a range from 48° C. to 52° C. is carried out;

and wherein in step (b) a heating to a temperature in a range from 82° C. to 97° C., preferably to a temperature in a range from 83° C. to 92° C., most preferably to a temperature in a range from 85° C. to 89° C. is carried out.

It is also possible in step (a) to increase the temperature to a value in a range from 82° C. to 97° C., preferably to a temperature in a range from 83° C. to 92° C., most preferably to a temperature in a range from 85° C. to 89° C. In preparation for this, it is possible to perform a step
(a1) mixing glycerol and cannabinoid, for example in the form of CBD oil, to produce a solution;
wherein in step (al) a heating to a temperature in a range from 82° C. to 97° C., preferably to a temperature in a range from 83° C. to 92° C., most preferably to a temperature in a range from 85° C. to 89° C. is carried out.

It is also possible, in preparation thereof, to perform a step
(a11) mixing water and at least one sucrose ester of an edible fatty acid,
wherein in step (a11) a heating to at least a temperature in a range from 40° C. to 62° C., preferably to a temperature in a range from 45° C. to 57° C., most preferably to a temperature in a range from 48° C. to 52° C. is carried out;
   and a step
(a12) addition of polysorbate 20 and/or polysorbate 80 to the mixture produced in step (a11) is carried out,
wherein in step (a12) a heating to a temperature in a range from 82° C. to 97° C., preferably to a temperature in a range from 83° C. to 92° C., most preferably to a temperature in a range from 85° C. to 89° C. is carried out.

Following step (a1) or following step (a11) or following step (a12), the cannabinoid is added in step (b).

### DETAILED DESCRIPTION

The invention will now be explained in more detail by way of exemplary embodiments. The following components were used:

### Curcumin

The product named “Turmeric Oleoresin Curcumin Powder 95%” with the product code EP-5001 from Green Leaf Extraction Pvt Ltd., Kerala, India, was used as the curcumin. The curcumin powder has CAS Number 458-37-7. It is a natural product obtained by solvent extraction of the rhizomes of Curcuma Longa. The curcumin content of the powder is at least 95%, according to manufacturer specifications. This curcumin content is determined by ASTA method 18.0.

As an alternative to the “oleoresin turmeric 95%” curcumin powder from Green Leaf mentioned above, it is also possible for the exemplary embodiments described below to use, as the curcumin, 95% curcumin extract by Neelam Phyto-Extracts, Mumbai, India, or curcumin BCM-95-SG or curcumin BCM-95-CG from eurochem GmbH, Gröbenzell, Germany, or Curcuma Oleoresin 95% from Henry Lamotte OILS GmbH, Bremen, Germany, for example.

### CBD

CBD oil was used as the source for a cannabinoid, in particular the product “CBD Drops PRM BLK 24%” from the manufacturer Pharmahemp as a first one. Furthermore, a CBD isolate with the brand name Cannapure® from the manufacturer Arevipharma GmbH was used, which contains at least 98% of synthetic cannabidiol.

### Boswellia

In the context of the present application, the term “Boswellia” in particular refers to an extract from the resin of the frankincense plant. Specifically, an extract of the species Boswellia serrata was used, which was an extract obtained by extraction with ethyl acetate from the resin of the plant with the botanical name Boswellia serrata with the product code “HC22519” manufactured by Frutarom Belgium N.V., Londerzeel, Belgium. A solubilizate containing this extract is also referred to as “boswellic acid solubilizate” because of its content of boswellic acids.

Besides extracts from the resin of the frankincense plant, it is also possible to use boswellic acids and/or derivatives of boswellic acids for the purposes of the solubilizates. In particular, the following may be considered: alpha-boswellic acid (CAS number 471-66-9), beta-boswellic acid (CAS number 631-69-6) and their derivatives, 3-O-acetyl-alpha-boswellic acid (CAS number 89913-60-0), 3-O-acetyl-beta-boswellic acid (CAS number 5968-70-7), 11-keto-beta-boswellic acid (KBA, CAS number 17019-92-0), and 3-O-acetyl-11-keto-beta-boswellic acid (AKBA, CAS number 67416-61-9).

### Xanthohumol

The products “Xantho-Flav” or “Xantho-Flav Pure” of the brand “Hopsteiner” by Simon H. Steiner, Hopfen, GmbH, Mainburg, Germany were used as the xanthohumol source. Both are natural products produced from hops. The active substance is the hop polyphenol xanthohumol. This is a yellow colored powder with a xanthohumol content between 65% and 85% in “Xantho-Flav” and at least 85% in “Xantho-Flav Pure”, according to manufacturer specifications.

The concentrations of xanthohumol and isoxanthohumol in “Xantho-Flav Pure” are quantified by the manufacturer according to UV spectrophotometric analysis or HPLC EBC 7.8 using external calibration standard pure XN (370 nm) or IX (290 nm). “Xantho-Flav Pure” contains the prenylated flavonoid xanthohumol in a very high concentration. For the exemplary embodiments in the context of the present application, “Xantho-Flav Pure” of batch number 9432 was used.

### Serrapeptase

The product named Serratiopeptidase 20,000 U/mg from Shaanxi Pioneer Biotech Co. Ltd. with batch number PBD 20170708 was used as the serrapeptase. This is a greyish white to light-brown powder. The enzyme unit (U) is a unit which has since been replaced by the katal to indicate enzymatic activity. Since the numerical values change when katal is used, the enzyme unit (U) continues to be used in medicine and clinical chemistry. One enzyme unit U corresponds to one micro-mole substrate conversion per minute.

### Polysorbate 80

The source of polysorbate 80 was the material “TEGO SMO 80 V FOOD” with the specification code “K04 EU-FOOD” from Evonik Nutrition & Care GmbH, Essen, Germany. The product complies with the EU requirements for food additive E 433. As an alternative to the TEGO SMO 80 V from Evonik mentioned above, it is also possible to use TEGO SMO 80 V from InCoPA Gmbh, Illertissen, Germany, or Crillet 4/Tween 80-LQ-(SG) from CRODA GmbH, Nettetal, Germany, or Lamesorb SMO 20 and Kotilen-O/1 VL from Univar or from Kolb Distributions AG, Hedingen, Switzerland, as the polysorbate 80 in the exemplary embodiments described below.

### Polysorbate 20

The source of polysorbate 20 was the material “TEGO SML 20 V FOOD” with the specification code “K09 EU-FOOD” from Evonik Nutrition & Care GmbH, Essen, Germany. The product complies with the EU requirements for food additive E 432. As an alternative to the TEGO SML 20 from Evonik mentioned above, it is also possible to use Crillet 1/Tween 20-LQ-(SG) from CRODA GmbH, Nettetal, Germany, as the polysorbate 20.

### Sucrose Esters of Edible Fatty Acids

A sucrose ester with the product designation “DUB SE 15 P” from the manufacturer Stearine Dubois was used as the sucrose ester of edible fatty acids. This ester is approved in the EU as food additive E473.

### Ethanol

In the context of the present application, ethanol was purchased from Berkel Pfälzische Spritfabrik GmbH & Co. KG. According to the specification for “undenatured neutral alcohol 1411U taxed”, the content of ethanol of this product is about 92.6 to 95.2 wt. %.

### Glycerol

The product used as glycerol in the context of the present application was “Glycamed 99.7%” from Glaconchemie GmbH, Merseburg, Germany. The glycerol content of this product is at least 99.5%, according to manufacturer specifications.

### Medium-Chain Triglycerides

Medium-chain triglycerides (MCTs) are triglycerides that contain medium-chain fatty acids. Medium-chain fatty acids include caproic acid, caprylic acid, capric acid and lauric acid. These are saturated fatty acids which naturally occur in tropical vegetable fats such as coconut oil and palm kernel oil. To a small extent they are also contained in milk fat. There is no pure MCT oil in nature, however, pure MCT oils can be obtained synthetically. Individual MCTs or a mixture of different MCTs can be used as medium-chain triglycerides. Medium-chain triglycerides were used in the form of MCT oil Delios VK Kosher, manufactured by Cognis GmbH, Monheim, Germany, or in the form of MCT oil (70/30) Rofetan GTCC 70/30 manufactured by DHW Deutsche Hydrierwerke Rodleben GmbH, Dessau-Roßlau, Germany, CAS number 73-398-61-5.

Furthermore, medium-chain triglycerides can be used in the form of the product ROFETAN DTCC 70/30 (Ph. Eur.). This is a caprylic/capric acid triglyceride with CAS number 73398-61-5. The product corresponds to the monograph “medium-chain triglycerides” of the European Pharmacopoeia valid at the filing date. Manufacturers are Ecogreen Oleochemicals DHW, Deutsche Hydrierwerke GmbH, Rodleben, Germany.

If water is added in the preparation of a solubilizate, distilled water is used.

The particle size analyzes of the micelles in aqueous dilutions of solubilizates were measured according to the principle of dynamic light scattering using laser light of 780 nm wavelength, unless stated otherwise. The particle size measurements were performed using the ParticleMetrix NANO-flex backscatter particle analyzer. The measuring principle is based on dynamic light scattering (DLS) in a 180° heterodyne backscattering setup.

For the experimental determination of turbidity of the solubilizates, the turbidity meters are calibrated with a standard suspension. Thus, instead of measured light intensity, the concentration of the calibration suspension is indicated. So, when any arbitrary suspension is measured, the indication means that the respective liquid causes the same light scattering as the standard suspension at the indicated concentration. The internationally defined turbidity standard is formazine. The most common units include the indication FNU, i.e. Formazin Nephelometric Units. This is the unit used in water treatment, for example, for measuring at 90° in compliance with the requirements of the ISO 7072 standard.

For preparing a solubilizate including the active substances curcumin and at least one cannabinoid as a further active substance it is possible to either mix individually prepared solubilizates with one another or to directly prepare a solubilizate that contains curcumin and at least one cannabinoid or several further active substances.

### Curcumin Solubilizates

By way of example, a 7% curcumin solubilizate is prepared. To this end,

| | |
|---|---|
| 925 g | polysorbate 80 and |
| 75 g | curcumin powder 95% (=71.2 g of curcumin) |

are used.

The polysorbate 80 is heated to between 48 and 52° C. The curcumin powder is added to the polysorbate under stirring, while further heating to a temperature in the range from 95 to 97° C. The powder is added at an appropriate rate so as to be evenly drawn into the emulsifier during stirring. After cooling to a temperature below a maximum of 60° C., the curcumin solubilizate is bottled. This solubilizate was used for the preparation of a curcumin and Boswellia solubilizate.

At a dilution ratio of 1:500 in water at pH 1.1 and a temperature of 37° C., the 7% curcumin solubilizate exhibits an average turbidity of 0.9 FNU.

However, it should be noted that the curcumin content can be further increased without having to accept adverse consequences, for example in terms of the stability of the micelles.

Also, the polysorbate 80 may be entirely or partially replaced by polysorbate 20 or by sucrose esters of edible fatty acids. For example, for preparing a curcumin solubilizate with polysorbate 20 alone, 894 g of polysorbate 80 and 106 g of 95% curcumin powder can be used. The polysorbate 20 is heated to between about 63° C. and about 67° C. While stirring, the curcumin powder is slowly added to the polysorbate 80. While adding the curcumin powder, heating is continued to between about 83° C. and about 87° C. The resulting solubilizate is slowly cooled to below about 45° C. and is then ready for being bottled.

Otherwise, the preparation of these variants corresponds to that described above. Solubilizates of up to about 11% can be produced in this way.

For preparing a 2.4% CBD solubilizate (free of water), the following is used:

| | |
|---|---|
| 900 g | polysorbate 80, and |
| 100 g | CBD oil (CBD Drops PRM BLK 24%, =24 g CBD). |

The polysorbate 80 is heated to between 48° C. and 52° C. The CBD oil is added to the polysorbate under stirring, while the temperature is further increased to a range between 85° C. and 89° C. After cooling to a temperature below a maximum of 60° C., the CBD oil solubilizate is bottled.

At a dilution ration of 1:50 in water at pH 1.1 and a temperature of 37° C., the anhydrous 2.4% CBD oil solubilizate exhibits a turbidity of 2.9 FNU.

An anhydrous CBD solubilizate, in particular one according to the above example, is particularly suitable as a filling for capsules.

A further example of a cannabinoid solubilizate is a 2.4% CBD solubilizate, for which the following is used:

| | |
|---|---|
| 100 g | CBD oil (CBD Drops PRM BLK 24%, =24 g CBD), |
| 27 g | sucrose ester of edible fatty acids, |
| 54 g | water, |
| 22.5 g | glycerol, and |
| 796.5 g | polysorbate 80. |

Water and glycerol are mixed and heated to a temperature in the range from 48° C. to 52° C. The sucrose ester is incorporated under vigorous stirring. The stirring is performed vigorously enough so that the sucrose ester dissolves in water and glycerol. Polysorbate 80 is added while stirring and is homogenized while heating to a temperature of 85° C. to 89° C. The stirring is performed vigorously enough so that the polysorbate 80 is evenly distributed. After having been cooled to a temperature below 60° C. while stirring, the CBD oil is incorporated under vigorous stirring and is completely homogenized while re-heating to a temperature of 85°° C. to 89° C. The stirring is performed vigorously enough so that the CBD oil is evenly distributed. The product is then cooled to a temperature below 60° C. and bottled. It is then stored in the dark at not more than 25° C.

At a dilution ration of 1:50 in water at pH 1.1 and a temperature of 37° C., the 2.4% CBD oil solubilizate exhibits a turbidity of 3.6 FNU. It is particularly suitable as an additive for beverages.

### 3% CBD Solubilisate (Free of Water)

The following is used:

| | |
|---|---|
| 31.2 g | CBD isolate (Cannapure ® at least 98%, =30.5 g CBD), |
| 62 g | glycerol, and |
| 906.8 g | polysorbate 80. |

Glycerol and CBD isolate are mixed together and heated to a temperature in the range from 85° C. to 89° C. The heating is conducted at a rate sufficient for the CBD to completely dissolve in the glycerol and to obtain a clear solution. Polysorbate 80 is added at a temperature between 85° C. and 89° C. while stirring. The stirring is performed vigorously enough so that the polysorbate 80 is evenly distributed. The product is cooled to a temperature below 60° C. and bottled. It is then stored in the dark at not more than 25° C.

At a dilution ration of 1:50 in water at pH 1.1 and a temperature of 37° C., the 2.4% CBD oil solubilizate exhibits a turbidity of 2.7 FNU. It is particularly suitable as a filling for capsules.

In particular for beverages, it is also possible to prepare the following 3% CBD solubilizate, with a reduction in the amount of polysorbate.

To this end, the following is used:

| | |
|---|---|
| 31.5 g | CBD isolate (Cannapure ® at least 98%, =30.5 g CBD),, |
| 27 g | sucrose ester of edible fatty acids, |
| 54 g | water, |
| 22.5 g | glycerol, and |
| 865 g | polysorbate 80. |

Water and sucrose ester are mixed together and heated to a temperature in the range from 48° C. to 52° C. Polysorbate 80 is added while stirring and is homogenized while heating to a temperature between 85° C. and 89° C. The stirring is performed vigorously enough so that the polysorbate 80 is evenly distributed. This is followed by cooling to a temperature below 60° C. while stirring. Separately, glycerol and the CBD isolate are mixed together and slowly heated to a temperature between 85° C. and 89° C. and completely homogenized, i.e. until a clear solution has been obtained. Subsequently, the polysorbate-sucrose ester mixture is incorporated into the CBD-glycerol mixture under vigorous stirring. While re-heating to a temperature between 85° C. and 89° C., stirring is performed vigorously enough so as to achieve an even distribution. The product is cooled to a temperature below 60° C. and bottled. It is then stored in the dark at not more than 25° C.

At a dilution ration of 1:50 in water at pH 1.1 and a temperature of 37°° C., this 3% CBD oil solubilizate exhibits a turbidity of 4.1 FNU. It is particularly suitable as an additive for beverages.

Besides the use of CBD oil, it is also possible to prepare and use cannabinoid solubilizates with THC oil and/or with oils or isolates or other sources of other cannabinoids as well, additionally or alternatively.

### 12% Boswellic Acid Solubilizate

The following is used:

| | |
|---|---|
| 76 g | 80% Boswellia serrata extract (=60.8 g boswellic acid), |
| 24 g | water, |
| 400 g | polysorbate 20. |

The water is mixed with the boswellia powder while being heated to a temperature in the range from 87 to 93° C. While maintaining the temperature, polysorbate 20 is incorporated. The emulsifier is added at such a rate, while stirring, that the fluids homogenize stably to form a solubilizate. Heavy foaming may occur during the preparation. This can be ignored if a clear solubilizate can be seen at the bottom of the collection vessel during bottling.

### 1.5% Serrapeptase Solubilizate

The following is used:

| | |
|---|---|
| 15 g | serrapeptase: serratiopeptidase 20,000 U/mg = 300,000,000 U, |
| 15 g | water, |
| 16.5 g | MCT oil, |
| 953.5 g | polysorbate 80. |

At a temperature in the range between 18 and 22° C., water is mixed with serrapeptase, and the mixture is homogenized. This means that the serrapeptase is distributed as evenly as possible in the water. This creates the conditions for the serrapeptase to be largely completely dissolved in the water. While heating to a temperature in the range from 83 to 87° C., MCT oil is incorporated into the water- serrapeptase mixture under constant stirring. The stirring is performed vigorously enough for the serrapeptase to dissolve evenly in the water. At unchanged temperature, polysorbate 80 is added under stirring and is homogenized. The stirring is performed vigorously enough for the polysorbate 80 to be evenly distributed. The product is cooled to a temperature below 60° C. and bottled. It is then stored in the dark at not more than 25° C.

300,000 U/g corresponds to 15 mg/g of 1.5% serrapeptase in enzymatic units. At a dilution in water of 1:50, the turbidity of this solubilizate was determined under physiological conditions at pH 1.1 and 37° C. The resulting value was 1.8 FNU.

10% Xantho-Flav Solubilizate (Corresponding to 7.5% Xanthohumol) with Ethanol

For this variant of a xanthohumol solubilizate, the following is used:

| | |
|---|---|
| 100 g | Xantho-Flav (\hat{=}75 g of xanthohumol), |
| 150 g | ethanol (96%) of neutral alcohol grade 1411 U, and |
| 750 g | polysorbate 80. |

First, the Xantho-Flav powder is dissolved in ethanol while being heated to a temperature in the range between 48 and 52° C. A homogeneous solution is created. Polysorbate 80 is then added into the solution of Xantho-Flav in ethanol while heating to between 83 and 87° C. The adding is done at a rate such that the two fluids homogenize well under stirring. The resulting solubilizate is cooled to below 60° C. and is bottled and stored in the dark and cool, i.e. at temperatures below 25° C.

The solubilizates described above can be used to prepare the solubilizate comprising curcumin and at least one further active substance by mixing. This will be described below with reference to exemplary embodiment 1.

In the context of the present application, a verification about whether the homogenization of the components to form a solubilizate has been sufficiently completed in the preparation of any solubilizates is obtained by measurements of the clarity of the product, which indicates complete micellization, using a laser beam. Such a laser beam measurement may be performed, for example, by illuminating the sample using a commercially available laser pointer, in particular with a wavelength in the range between 650 nm and 1700 nm (spectral color red), and subsequent visual inspection of the illuminated or irradiated solubilizate. The verification is not achieved by sampling and thus outside the reaction vessel, but in the reaction vessel. The laser beam is directed through a sight glass which is located on the front of the reaction vessel, perpendicularly to the reaction vessel. If merely a point of light appears on the rear inner surface of the reaction vessel, completely free of scattering, the resulting particle structures in the reaction vessel are smaller than the wavelength of the visible light, which is thus a visual confirmation that the process of micellization has been completed.

### EXAMPLE 1

Solubilizate of 1.4% Curcumin/2.4% Boswellic Acid/1.5% Xanthohumol/0.48% CBD/0.3% Serrapeptase

The following is used according to the respective formulations as described above:

| | |
|---|---|
| 200 g | 7% curcumin solubilizate, |
| 200 g | 12% Boswellia solubilizate, |
| 200 g | 7.5% xanthohumol solubilizate, and |
| 200 g | 2.4% water-free CBD solubilizate, |
| 200 g | 1.5% serrapeptase solubilizate. |

All five solubilizates can be heated to a temperature in the range from 50° C. to 60° C. to lower their viscosity and thus enhance flowability. Then, they are mixed together by stirring. Once a homogeneous complete product is obtained, it is optionally cooled to a temperature below 60° C. and bottled.

Prior to further processing such as filling into capsules, it is favorable to again stir the product to homogenize it, and if necessary to this end to heat it moderately, i.e. to a temperature of about 40°° C. to 50° C.

At a dilution ratio of 1:50 in water at a pH of 1.1 and a temperature of 37°° C., the solubilizate exhibits an averaged turbidity of 3.4 FNU.

### EXAMPLE 2

### Directly Preparated Solubilizate of 1.4% Curcumin/2.4% Boswellic Acids/1.5% Xanthohumol/0.048% CBD/3% Serrapeptase

The following is used:

| | |
|---|---|
| 15 g | 95% curcumin powder, |
| 30.4 g | Boswellia Serrata extract (24.3 boswellic acid), |
| 20 g | Xantho-Flav powder (at least 70% xanthohumol = 15 g |
| | xanthohumol), |
| 20 g | CBD oil (0.48 g CBD), |
| 3 g | serrapeptase (60,000,000 U), |
| 12.6 g | water, |
| 20 g | ethanol, |
| 3.3 g | MCT oil, |
| 160 g | polysorbate 20, and |
| 715.7 g | polysorbate 80. |

Polysorbate 80 and polysorbate 20 are mixed at a temperature in the range between 48° C. and 52° C. while stirring vigorously enough so that a homogeneous mixture is obtained. Water and ethanol are added while stirring vigorously enough so that a homogeneous mixture is obtained at a temperature in the range from 48° C. to 52°° C. Xanthohumol and Boswellia are slowly admixed under consistent stirring. The temperature is then increased to 63° C. to 67° C. under vigorous stirring. Subsequently, curcumin, serrapeptase, CBD oil, and MCT oil are incorporated into the mixture. Care is taken to ensure good mixing and homogeneity of the product. If appropriate, pauses are taken between the addition of a substance and the addition of the next substance. The adding is conducted slowly enough, while stirring, so that the particular ingredient to be added is evenly incorporated into the preparation.

The temperature is further increased to a value in the range between 85° C. and 89°° C. The heating is conducted under constant stirring and slowly enough to ensure that the mixture that is being heated remains homogeneous and so that a clear product is obtained. After cooling to a temperature below 60° C., the product is bottled. It is dark and viscous and is stored in the dark at temperatures of not more than 25° C. Prior to further processing such as filling into capsules, it is favorable to again stir the product to homogenize it and, if necessary to this end, to heat it moderately, i.e. to a temperature of about 40° C. to 50° C.

At a dilution ratio of 1:50 in water at a pH of 1.1 and a temperature of 37° C., the solubilizate exhibits a turbidity of 3.4 FNU.

### EXAMPLE 3

### Solubilizate of 3% Curcumin/3.2% Boswellic Acid/1.6% Xanthohumol/1% CBD Oil

The following is used:

| | |
|---|---|
| 40.5 g | 80% Boswellia serrata extract (32.4% boswellic acid), |
| 31.5 g | 95% curcumin powder (29.925 g of curcumin), |
| 20.7 g | Xantho-Flav powder containing at least 80% of |
| | xanthohumol (16.5 g xanthohumol), |
| 54.3 g | water, |
| 45 g | ethanol, |
| 315 g | polysorbate 20, |
| 483 g | polysorbate 80, |
| 10 g | CBD oil: 30% cannabidiol. |

Cannabidiol (CBD) is a barely psychoactive cannabinoid derived from the female hemp Cannabis sativa or Cannabis indica. A non-THC-free CBD oil was used, which means that it may contain traces of THC. Tetrahydrocannabinol (THC) is responsible for the psychedelic effects of hemp plants.

While heating to a temperature in the range from 48 to 52° C., polysorbate 20 and polysorbate 80 are homogenized with each other while being dissolved in each other, under stirring. While maintaining the temperature, the emulsifier mixture is mixed with the water and ethanol, while stirring is performed vigorously enough so that the water and the ethanol are dissolved evenly in the emulsifier solution. At unchanged temperature, the Boswellia serrata extract and the xanthohumol are incorporated into the water-diluted emulsifier mixture while stirring. The adding is performed at a rate slow enough so that the Boswellia serrata extract and the xanthohumol are evenly drawn into the dilute emulsifier solution under stirring. Subsequently, the temperature is increased to a range between 63° C. and 67° C. under vigorous stirring. The curcumin powder is incorporated while stirring. The temperature is further increased to a value in the range between 85° C. and 89° C. while stirring vigorously enough so that the curcumin is evenly distributed in the preparation and homogenized. Subsequently, the CBD oil is incorporated into the mixture while stirring vigorously enough so that the CBD oil is evenly distributed in the preparation and homogenized.

This is followed by cooling to a temperature of less than or equal to 45° C. The dark-yellow viscous preparation comprising a solubilizate of curcumin and boswellic acid and xanthohumol and CBD oil is then bottled and stored in the dark and cool, i.e. below 25° C. The solubilizate and an aqueous solution thereof are stably homogeneous and soluble in crystal clear form.

It will be apparent to a person skilled in the art that the invention is not limited to the examples described above, but rather can be varied in various ways. It is in particular possible to combine or swap the features of the individually illustrated examples. This applies in particular to the emulsifier composition and the composition of active substances of the solubilizate according to the invention.

What is claimed is:

1. A solubilizate, consisting of:
a cannabinoid in an active amount between 0.3 wt. % and 10 wt. %;
an emulsifier selected from the group consisting of polysorbate 80, a mixture of polysorbate 80 and polysorbate 20, and a mixture of polysorbate 80 and a sucrose ester of edible fatty acids, in an amount of at least 70 wt. %;
0-20 wt. % ethanol;
0-25 wt. % glycerol; and
0-10 wt. % water.

2. The solubilizate as in claim 1,
wherein the active amount of the cannabinoid is between 0.3 wt. % and 3 wt. %.

3. The solubilizate as in claim 1,
wherein a diameter distribution of micelles in a dilution of the solubilizate with distilled water in a ratio of 1:500 under physiological conditions (pH 1.1 and 37° C.) extends from d10 =6 nm to d90 =20 nm.

4. The solubilizate as in claim 1,
wherein a turbidity of the solubilizate is less than 25 FNU, measured by scattered light measurement using infrared light according to the specifications of the ISO 7027 standard at a dilution of the solubilizate in a ratio of 1:50 or 1:500 in water under physiological conditions (pH 1.1 and 37° C.).

5. The solubilizate as in claim 1,
wherein the cannabinoid is CBD.

6. The solubilizate as in claim 1.
wherein the cannabinoid is THC.

7. The solubilizate as in claim 1.
wherein the solubilizate provides oxidative protection of the cannabinoid.

8. A mixture, comprising:
the solubilizate as in claim 1, and
a further solubilizate.

9. A capsule, comprising:
the solubilizate as in claim 1.
